(19) United States
(12) Patent Application Publication
(10) Pub. No.: US 2006/0004209 A1
(43) Pub. Date: Jan. 5, 2006

(54) Process for preparing highly pure and free-flowing solid of 7-ethyltryptophol

(76) Inventors: Taesoo Kwon, Montville, NJ (US); Hanrong Gao, Lake Hiawatha, NJ (US); Satish Bodige, Highland, NJ (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/883,957

(22) Filed: Jul. 2, 2004

(51) Int. Cl. C07D 209/14

(52) U.S. Cl. 548/504

(57) ABSTRACT

An industrial purification process for preparing a highly pure and free flowing solid of 7-ethyltryptophol. Crude 7-ethyltryptophol, prepared by, known procedures is dissolved in an organic solvent and washed with aqueous acid solution to form an aqueous phase and an organic phase. After at least partially removing the solvent from the organic phase, it is triturated with an alkane solvent under cooling to solidify the residue. A highly pure and free-flowing solid of 7-ethyltryptophol is recovered therefrom.

### RELATED APPLICATIONS

This application is a continuation of U.S. provisional Application Ser. No. 60/434,598, filed Jul. 2, 2003.

### FIELD OF THE INVENTION

This invention relates to the purification of an important chemical intermediate.

### BACKGROUND OF THE INVENTION

In accordance with the present invention, there is disclosed a purification process for the preparation of a highly pure and free-flowing solid of 7-ethyltryptophol from the crude tarry 7-ethyltryptophol. 7-ethyltryptophol is the key intermediate of a potent antiinflammatory and analgesic compound, Etodolac. Etodolac is a pyranocarboxylic acid, chemically designated as (±) 1,8-diethyl-1,3,4,9-tetrahydropyrano-[3,4-b]indole-1-acetic acid.

Two methods for the preparation of 7-ethyltryptophol are disclosed in U.S. Pat. No. 4,585,877 to Demerson et al. and PCT Publication WO 99/59,970 to Stevensen et al. in which 7-ethyltryptophol was prepared by the Fischer indole synthesis reaction of 2-ethylphenylhydrazine hydrochloride and 2,3-dihydrofuran. A similar process of making 7-ethyltryptophol by the reaction of 2-ethylphenylhydrazine hydrochloride and 4-hydroxybutyraldehyde is disclosed in U.S. Pat. No. 4,012,417 to Demerson et al.

Demerson et al., J. Med. Chem., 391(1976) also discloses the preparation of 7-ethyltryptophol by the reduction of 7-ethyl-3-indolyglyoxylate with LiAlH₄. The glyoxylate is produced by the reaction of 7-ethylindole with oxalyl chloride, and the 7-ethylindole is made from 2-ethylaniline in a three-step process.

The method of making 7-ethyltryptophol by the reaction of 2-ethylphenylhydrazine hydrochloride and 2,3-dihydrofuran is cheaper and simpler than other methods. However, the reaction is not clean and produces a lot of impurities. The 7-ethyltryptophol isolated from the reaction is a tarry solid or a sticky oil with a low purity unless flash column chromatography on silica gel was used for the purification (see U.S. Pat. No. 4,585,877 to Demerson et al. and WO 99/59.970 to Sevensen et al.).

Purification by column chromatography is not economical and very difficult to implement in industrial manufacture. Although other purification methods such as crystallization and washing of a suspension or solution, are suitable ways to purify the products in manufacture production, to the best of our knowledge, no process for making highly pure and free-flowing solid of 7-ethyltryptophol has been developed so far except by using flash column chromatography.

Highly pure and free-flowing solid of 7-ethyltryptophol is desirable for the preparation of etodolac in the terms of both convenience of material handling and product purity. There is thus a need for a simple and economical chemical process for making pure and free-flowing solid of 7-ethyltryptophol.

### SUMMARY OF THE INVENTION

According to the process of the present invention, Crude 7-ethyltryptophol is purified by dissolving the crude 7-ethyltryptophol (which may be a tarry solid or a sticky oil) in an organic solvent, followed by washing with an aqueous solution of acid at ambient temperature. After removing part or all of the solvent, preferably by distillation, the product is solidified in an alkane solvent under cooling. The process allows the formation of a free-flowing solid with an increased HPLC purity and assay.

### DETAILED DESCRIPTION OF THE INVENTION

Crude tarry 7-ethyltryptophol prepared by known methods contains many polar and non-polar impurities. In accordance with the present invention, there is provided a novel process for making a highly pure and free-flowing solid of 7-ethyltryptophol from the crude tarry solid or sticky oil product. This provides an excellent intermediate for further synthesis, eliminating the preheating operation necessary for handling the tarry 7-ethyltryptophol and resulting in a high purity product which is useful as an intermediate for further synthesis. The process comprises dissolving crude 7-ethyltryptophol prepared by the known methods in an organic solvent, dissolution of the crude preparation may be carried out at ambient temperature, and washing the resultant solution with an aqueous acid solution to remove most of the impurities, which are separated into the resulting aqueous fraction and removed. This is followed by removal of part or all of the organic solvent and solidification of the remaining residue in an alkane solvent. In preferred embodiments, all or substantially all of the organic solvent is removed via distillation and solidification of the remaining residue is accomplished via trituration in the alkane solvent under cooling with vigorous stirring.

The organic solvent utilized to dissolve the crude product can be an solvent in which 7-ethyltryptophol is soluble. Preferably, the solvent is one that has a good solubility for 7-ethyltryptophol at ambient temperature and good layer separation with aqueous acid solution. Examples of suitable organic solvents which can be useful to dissolve the crude 7-ethyltryptophol include, without intended limitation, dichloromethane, t-butyl methyl ether, diethyl ether, toluene, 1,2-dichloroethane, benzene, ethyl acetate and t-butyl acetate. The amount of the organic solvent used in the purification is at least sufficient to completely dissolve the crude 7-ethyltryptophol, but preferably the organic solvent may be present in greater than equimolar amounts.

The aqueous solutions of acids used for the washing step include, without intended limitation, inorganic acids such as hydrochloric acid, sulfuric acid, nitric acid and phosphoric acid and suitable organic acids, such as acetic acid. The concentration of the aqueous acid solution can be in the range of 0.1 to 10 N, preferably, from about 1 N to 2 N. The washing to remove impurities is carried out by mixing the solution of 7-ethyltryptophol and the aqueous acid solution and stirring at a temperature lower then 100° C. or the boiling point of the organic solvent. However, in order to avoid any reaction between 7-ethyltryptophol and the acid, the washing is preferrably carried out at a temperature from about 10° C. to 25° C. When said preferred temperature range, is employed, the stirring time is typically about 30 to 60 min for each washing.

The number of washings and the time of each will depend on 1) the volume and concentration of the aqueous acid solution used for each washing: and 2) the concentration of 7-ethyltryptophol in the organic solution. After washing and removal of the aqueous phase, the organic solvent is removed from the resultant Solution of 7-tryptophol. Preferably this is accomplished via distillation under vacuum to remove from about 50-100% of the organic solvent, preferably removing as much as possible. In most preferred embodiments, all or substantially all of the organic solvent is removed via distillation. The distillation temperature should be higher than 50° C. if the solvent is completely removed. At this temperature, the 7-ethyltryptophol is in a melted state and therefore, stirring will not be impeded during the distillation.

Solidification of the 7-ethyltryptophol is preferably performed by triturating the resultant residue with an alkane solvent to at a temperature of 50° C. or higher but lower than the boiling point of the alkane solvent. The most commonly employed alkane solvents for the solidification are pentane, hexane, cyclohexane, heptane and petroleum ether, but any of other alkane solvents can be used as well. Preferably, the mixture is slowly cooled under vigorous stirring to 5° C. or lower, preferably from about −20 to 5° C., and then stirring is continued for one to two hours. The resultant mixture is filtered and the recovered solid is washed with the alkane solvent and then dried under a vacuum at ambient temperature. The obtained solid is free-flowing and its HPLC purity and assay are usually 20% higher than those of the crude product.

The following examples serve to illustrate the present invention and are not in any way, to be considered as a limitation thereof.

### EXAMPLE 1

Two hundred grams of crude 7-ethyltryptophol, which has an HPLC purity and assay of 83.0% and 75.4% respectively, were dissolved in 1400 mL of dichloromethane at room temperature. The solution was successively washed at room temperature with three 600 mL portions of 1 N aqueous solution of HCl, 600 mL at saturated sodium bicarbonate solution and 400 mL of brine. After completely removing the solvent by evaporating on a rotary vapor at 50˜55° C. 350 mL of hexane was added at 50˜55° C. under vigorous stirring. The mixture was then slowly cooled to 0˜5° C. over 1 h and stirred at 0˜5° C. for another 2 h. After filtering, the solid was washed with 200 ml of hexane and dried at 25˜30° C. under vacuum overnight to give 164 g (82% recovery) of the solid product. HPLC purity and assay: 96.2% and 88.4%, respectively.

### EXAMPLE 2

Two hundred grams of crude 7-ethyltryptophol, which had an HPLC purity and assay of 76.7% and 62.2%, respectively, were dissolved in 1400 mL of t-butyl methyl ether at room temperature. The solution was successively washed at room temperature with three 600 mL portions of 1 N aqueous solution of HCl, 600 mL of saturated sodium bicarbonate solution and 400 mL of brine. After completely removing the solvent by evaporating on a rotary vapor at 50˜55° C. 350 mL of hexane was added at 50˜55° C. under vigorous stirring. The mixture was then slowly cooled to 0˜5° C. over 1 hour and stirred at 0—5° C. for another 2 hours. After filtering, the solid was washed with 200 mL of hexane and dried at 25˜30° C. under vacuum overnight to give 168 g (84% recovery) of the solid product. HPLC purity and assay: 96.9% and 86%, respectively.

### EXAMPLE 3

Twenty grams of crude 7-ethyltryptophol, which had an HPLC purity and assay of 76.7% and 62.2%, respectively, were dissolved in 150 mL of toluene at room temperature. The solution was successively washed at room temperature with three 80 mL portions of 1 N aqueous solution of HCl, 80 mL of saturated sodium bicarbonate solution and 60 mL of brine. After evaporating on a rotary vapor at 50° C. to a volume of 30˜35 mL, 60 mL of hexane was added and stirred at refluxing for 20 min. The mixture was then slowly cooled to −10° C. over 1 h and stirred at −10° C. for another 2 h. After filtering, the solid was washed with 20 mL of hexane and dried at 25˜30° C. under vacuum overnight to give 11.2 g (56% recovery) of the solid product HPLC purity and assay: 95.3% and 87.2%, respectively.

What is claimed is:

1. A process for preparing highly pure 7-ethyltryptophol, comprising:
a) dissolving crude 7-ethyltryptophol in an organic solvent in which 7-ethyltryptophol is soluble;
b) treating the solution formed in step (a) b addition of an aqueous acid solution to form separate aqueous and organic phases:
c) separating said aqueous phase from said organic phase:
d) at least partially removing said organic solvent from said organic phase:
e) solidifying the residue produced in step (d) by adding an alkane solvent: and
f) recovering highly pure solid 7-ethyltryptophol from the mixture of step (e).

2. The process in accordance with claim 1, wherein said organic solvent in step (a) is selected from the group consisting of chloroform, dichloromethane, toluene, benzene, t-butyl methyl ether, diethyl ether, dioxane, ethyl acetate and isopropyl acetate.

3. The process in accordance with claim 1, wherein said aqueous acid solution added in step (b) comprises an acid selected from the group consisting of hydrochloric acid, sulfuric acid, nitric acid, phosphoric acid and acetic acid.

4. The process in accordance with claim 1, wherein the concentration of said aqueous acid solution added in step (b) is from about 0.1 N to about 10 N.

5. The process in accordance with claim 1, wherein the treatment with aqueous acid solution in step b) is conducted at a temperature of from about 10° C. to about 50° C.

6. The process in accordance with claim 1, wherein the treatment with aqueous acid solution at step b) comprises mixing and stirring together said organic solution and said aqueous acid solution such that at least a majority of impurities present in the mixtures are contained in said separate aqueous phase.

7. The process in accordance with claim 1, wherein at the organic solvent is removed in step d) by distillation.

8. The process in accordance with claim 1, wherein the alkane solvent added to the residue in step e) has a low solubility for 7-ethyltryptophol.

9. The process in accordance with claim 8, wherein said alkane solvent is selected from the group consisting of pentane, hexane, heptane, cyclohexane and petroleum ether.

10. The process in accordance with claim 1, wherein said solidification in step (e) comprises trituration conducted under cooling conditions.

11. The process in accordance with claim 10, wherein said cooling conditions comprise lowering the temperature during solidification to a range of about −20 to 5° C.

12. The process in accordance with claim 1, wherein said highly pure solid 7-ethyltryptophol is recovered from the mixture of step e) by filtration.

13. The process in accordance with claim 1 wherein said crude 7-ethyltryptophol comprises a tarry solid, or a sticky oil.

14. The process in accordance with claim 1, wherein the treatment in said organic solvent in step a) is conducted at ambient temperature.
